(19) United States
(12) Patent Application Publication
(10) Pub. No.: US 2019/0239859 A1
(43) Pub. Date: Aug. 8, 2019

(54) MEDICAL IMAGE DIAGNOSTIC APPARATUS AND X-RAY IRRADIATION CONTROLLER

(71) Applicant: CANON MEDICAL SYSTEMS CORPORATION, Otawara-shi (JP)

(72) Inventors: Sayaka TAKAHASHI, Otawara (JP); Ryoichi Nagae, Nasushiobara (JP); Nobuhide Ooi, Nasushiobara (JP); Mitsuo Akiyama, Otawara (JP); Koji Ando, Otawara (JP); Minori Izumi, Shioya (JP); Takashi Koyakumaru, Utsunomiya (JP)

(73) Assignee: CANON MEDICAL SYSTEMS CORPORATION, Otawara-shi (JP)

(21) Appl. No.: 16/261,624

(22) Filed: Jan. 30, 2019

(30) Foreign Application Priority Data
Feb. 2, 2018 (JP) 2018-017193

(51) Int. Cl. A61B 8/08; A61B 8/00; A61B 5/055; A61B 6/03; A61B 6/00

(52) U.S. Cl. CPC A61B 8/5261; A61B 8/463; A61B 5/055; A61B 6/032; A61B 8/465; A61B 6/463; A61M 2025/0166; A61B 6/5247; A61B 8/54; A61B 6/54; A61B 6/566; A61B 8/565; A61B 6/563; A61B 6/465

(57) ABSTRACT

The medical image diagnostic apparatus according to a present embodiment includes processing circuitry. The processing circuitry is configured to display an ultrasonic image and a non-ultrasonic medical image to a display. The processing circuitry is configured to determine which of a displayed ultrasonic image and a displayed non-ultrasonic medical image is live. The processing circuitry is configured to inform information indicating which any one of the displayed ultrasonic image and the displayed non-ultrasonic medical image is live in accordance with the determination.

### CROSS-REFERENCE TO RELATED APPLICATION

This application is based upon and claims the benefit of priority from Japanese Patent Application No. 2018-017193, filed on Feb. 2, 2018, the entire contents of each of which are incorporated herein by reference.

### FIELD

An embodiment as an aspect of the present invention relates to a medical image diagnostic apparatus and an X-ray irradiation controller.

### BACKGROUND

There are medical image diagnostic systems each having different medical image diagnostic apparatuses for the purpose of improving treatment efficiency by using different kinds of apparatuses such as an X-ray diagnostic apparatus, an ultrasonic diagnostic apparatus, an X-ray computed Tomography (CT) apparatus, and a magnetic resonance imaging apparatus in combination. For example, when an interventional treatment using a catheter is performed, the X-ray diagnostic apparatus and the ultrasonic diagnostic apparatus are used in combination.

The X-ray diagnostic apparatus transmits X-rays into an object and images the transmitted X-rays. As means for acquiring X-ray projection images, there are “radiography mode” which irradiates relatively strong X-rays and “fluoroscopy mode” which irradiates relatively weak X-rays. A doctor inserts the catheter into a patient while confirming the catheter in the blood vessel by X-ray irradiation in the radiography mode or the fluoroscopy mode. After reaching the affected part of the catheter, imaging of the affected part is performed from any angles by X-rays. Thereafter, the identified affected part is treated with the catheter. A method of specifying the affected part by using the ultrasonic diagnostic apparatus in combination is attracting attention so as not to overlook lesions which cannot be confirmed by the radiography and the photography by the X-rays.

In a case of catheter treatment for pediatric patients, the dose and irradiation time of the X-rays must be particularly kept lower than in the case of adult patients in order to avoid exposure. Even in such a case, the combined use of the X-ray diagnostic apparatus and the ultrasonic diagnostic apparatus is effective.

### BRIEF DESCRIPTION OF THE DRAWINGS

FIG. 1 is a schematic diagram showing a configuration of a medical image diagnostic system according to a first embodiment.

FIG. 2 is a diagram showing an appearance of the medical image diagnostic system according to the first embodiment.

FIG. 3 is a flowchart showing a first operation example of the medical image diagnostic system according to the first embodiment.

FIG. 4 is a diagram showing an example of a superimposed image in which live information is given as the informing image in the medical image diagnostic system according to the first embodiment.

FIG. 5 is a flowchart showing a second operation example of the medical image diagnostic system according to the first embodiment.

FIG. 6 is a diagram showing an example of a superimposed image in which live information is given as the informing image in the medical image diagnostic system according to the first embodiment.

FIG. 7 is a schematic diagram showing a configuration of a medical image diagnostic system according to a second embodiment.

FIG. 8 is a schematic diagram showing a configuration of a medical image diagnostic system according to a third embodiment.

### DETAILED DESCRIPTION

A medical image diagnostic apparatus and an X-ray irradiation controller according to a present embodiment will be described with reference to the accompanying drawings.

The medical image diagnostic apparatus according to a present embodiment includes processing circuitry. The processing circuitry is configured to display an ultrasonic image and a non-ultrasonic medical image to a display. The processing circuitry is configured to determine which of a displayed ultrasonic image and a displayed non-ultrasonic medical image is live. The processing circuitry is configured to inform information indicating which any one of the displayed ultrasonic image and the displayed non-ultrasonic medical image is live in accordance with the determination.

In the medical image diagnostic apparatus according to the embodiment, the non-ultrasonic medical image is, for example, an X-ray CT (Computed Tomography) image, an MR (Magnetic Resonance) image, an X-ray projection image, or the like. When the non-ultrasonic medical image is the X-ray CT image, the medical image diagnostic system according to the embodiment includes at least an ultrasonic diagnostic apparatus and an X-ray CT apparatus. When performing interventional treatment using a catheter, this medical image diagnostic system makes full use of the ultrasonic image obtained from the ultrasonic diagnostic apparatus and the X-ray CT image which is a cross sectional image obtained from the X-ray CT apparatus, and then the procedure to the patient to be described later is advanced.

When the non-ultrasonic medical image is the MR image, the medical image diagnostic system according to the embodiment includes at least the ultrasonic diagnostic apparatus and an MRI (Magnetic Resonance Imaging) apparatus. When performing interventional treatment using a catheter, this medical image diagnostic system makes full use of the ultrasonic image obtained from the ultrasonic diagnostic apparatus and the MR image which is a cross sectional image obtained from the MRI apparatus, and then the procedure to the patient to be described later is advanced. In order to facilitate a procedure for the patient, it is preferable that the MRI apparatus is of open type in which a pair of magnets is disposed above and below an imaging space.

When the non-ultrasonic medical image is the X-ray projection image, the medical image diagnostic system according to the embodiment includes at least the ultrasonic diagnostic apparatus and an X-ray diagnostic apparatus. When performing interventional treatment using a catheter, this medical image diagnostic system makes full use of the ultrasonic image obtained from the ultrasonic diagnostic apparatus and the X-ray projection image obtained from the X-ray diagnostic apparatus, and then the procedure to the patient to be described later is advanced.

In the case where the non-ultrasonic medical image is the X-ray projection image, the medical image diagnosis system according to the embodiment may be provided with the X-ray CT apparatus instead of the X-ray diagnosis apparatus. This is because the X-ray CT apparatus can acquire the X-ray projection image by an imaging method with the rotation of the X-ray tube stopped. This imaging method is also called “CT fluoroscopy”.

Hereinafter, the medical image diagnostic system according to the embodiment includes at least the ultrasonic diagnostic apparatus and the X-ray diagnostic apparatus, and a case where the ultrasonic image obtained from the ultrasonic diagnostic apparatus and the X-ray projection image obtained from the X-ray diagnostic apparatus are used will be described as an example.

1. Medical Image Diagnostic System

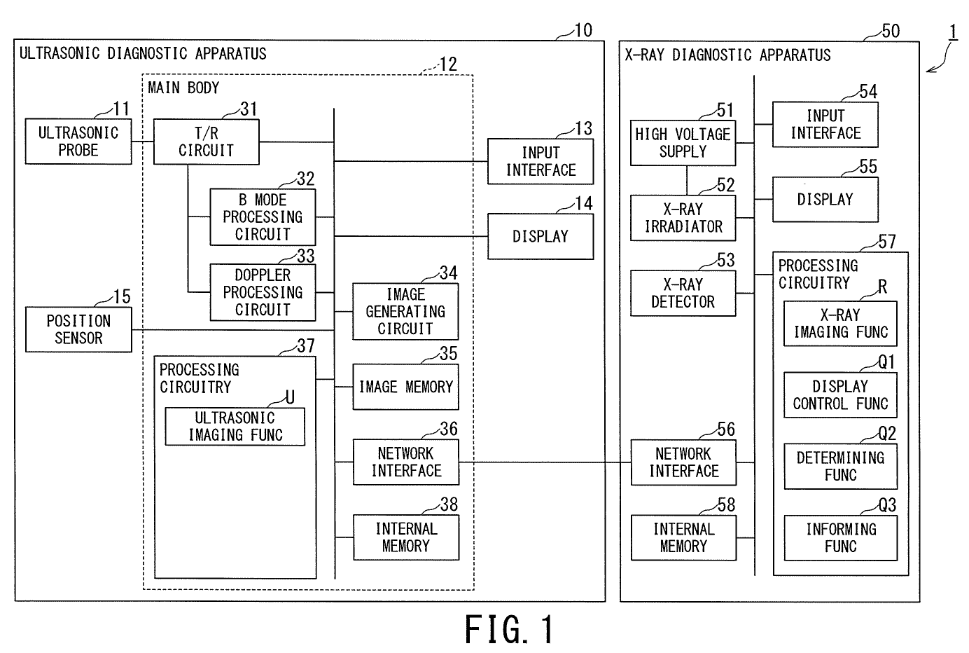

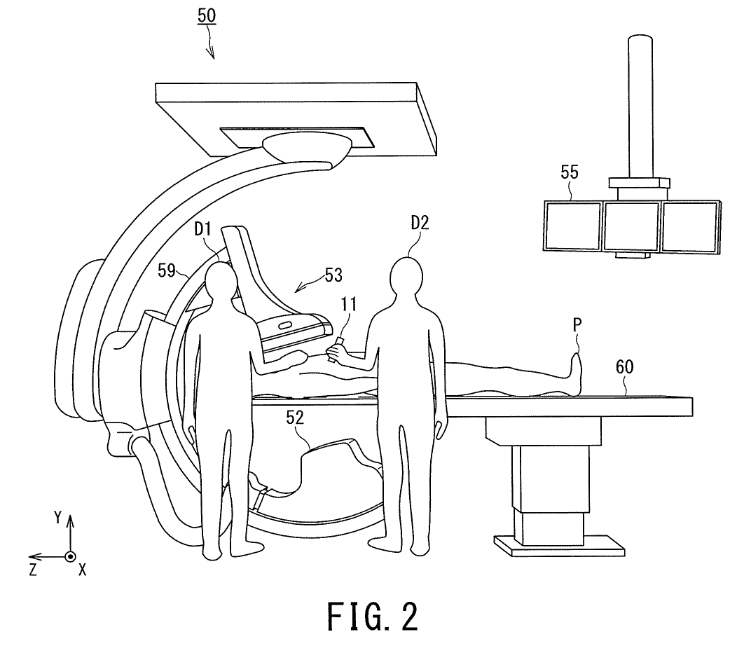

FIG. 1 is a schematic diagram showing a configuration of a medical image diagnostic system according to a first embodiment. FIG. 2 is a diagram showing an appearance of the medical image diagnostic system according to the first embodiment.

FIGS. 1 and 2 show a medical image diagnostic system 1 according to a first embodiment. The medical image diagnostic system 1 includes an ultrasonic diagnostic apparatus 10 and an X-ray diagnostic apparatus 50 as a medical image diagnostic apparatus according to a first embodiment. For example, the X-ray diagnostic apparatus 50 is an X-ray cardiovascular apparatus, so-called an angio apparatus.

The ultrasonic diagnostic apparatus 10 includes an ultrasonic probe 11, a main body 12, an input interface 13, and a display 14. It should be noted that a configuration of the main body 12 alone may be referred to as an ultrasonic diagnostic apparatus in some cases. Alternatively, a configuration in which at least one of the ultrasonic probe 11, the input interface 13, and the display 14 is added to the main body 12 is sometimes referred to as an ultrasonic diagnostic apparatus. In the following description, the case where the configuration including all of the ultrasonic probe 11, the main body 12, the input interface 13, and the display 14 in an ultrasonic diagnostic apparatus will be described.

The ultrasonic probe 11 includes microscopic transducers (piezoelectric elements) on the front surface portion, and transmits and receives ultrasonic waves to a region including a scan target, for example, a region including a lumen. Each transducer is an electroacoustic transducer, and has a function of converting electric pulses into ultrasonic pulses at the time of transmission and converting reflected waves to electric signals (reception signals) at the time of reception. The ultrasonic probe 11 is configured to be small and lightweight, and is connected to the main body 12 via a cable (or wireless communication).

The ultrasonic probe 11 is classified into types such as a linear type, a convex type, a sector type, etc., depending on differences in scanning system. The ultrasonic probe 11 is classified into a 1D array probe in which transducers are arrayed in a one-dimensional (1D) manner in the azimuth direction, and a 2D array probe in which transducers are arrayed in two dimensions (2D) manner in the azimuth direction and in the elevation direction, depending on the array arrangement dimension. The 1D array probe includes a probe in which a small number of transducers are arranged in the elevation direction.

In this embodiment, when a 3D scan, that is, a volume scan is executed, the 2D array probe having a scan type such as the linear type, the convex type, the sector type, or the like is used as the ultrasonic probe 11. Alternatively, when the volume scan is executed, the 1D probe having a scan type such as the linear type, the convex type, the sector type and the like and having a mechanism that mechanically oscillates in the elevation direction is used as the ultrasonic probe 11. The latter probe is also called a mechanical 4D probe.

The main body 12 includes a transmitting and receiving (T/R) circuit 31, a B mode processing circuit 32, a Doppler processing circuit 33, an image generating circuit 34, an image memory 35, a network interface 36, processing circuitry 37, and an internal memory 38. The circuits 31 to 34 are configured by an application specific integrated circuit (ASIC) or the like. However, the present invention is not limited to this case, and all or a part of the functions of the circuits 31 to 34 may be realized by the processing circuitry 37 executing a program.

The transmitting and receiving circuit 31 has a transmitting circuit and a receiving circuit (not shown). Under the control of the processing circuitry 37, the transmitting and receiving circuit 31 controls transmission directivity and reception directivity in transmission and reception of ultrasonic waves. The case where the transmitting and receiving circuit 31 is provided in the main body 12 will be described, but the transmitting and receiving circuit 31 may be provided in the ultrasonic probe 11, or may be provided in both of the ultrasonic diagnostic apparatus 10 and the main body 12.

The transmitting circuit has a pulse generating circuit, a transmission delay circuit, a pulsar circuit and the like, and supplies a drive signal to ultrasonic transducers. The pulse generating circuit repeatedly generates a rate pulse for forming a transmission ultrasonic wave at a predetermined rate frequency. The transmission delay circuit converges the ultrasonic waves generated from the ultrasonic transducer of the ultrasonic probe 11 into a beam shape, and gives a delay time for each piezoelectric transducer necessary for determining the transmission directivity to each rate pulse generated by the pulse generating circuit. In addition, the pulsar circuit applies a drive pulse to the ultrasonic transducers at a timing based on the rate pulse. The transmission delay circuit arbitrarily adjusts the transmission direction of the ultrasonic beam transmitted from a piezoelectric transducer surface by changing the delay time given to each rate pulse.

The receiving circuit has an amplifier circuit, an A/D (Analog to Digital) converter, an adder, and the like, and receives the echo signal received by the ultrasonic transducers and performs various processes on the echo signal to generate echo data. The amplifier circuit amplifies the echo signal for each channel, and performs gain correction processing. The A/D converter A/D-converts the gain-corrected echo signal, and gives a delay time necessary for determining the reception directivity to the digital data. The adder adds the echo signal processed by the A/D converter to generate echo data. By the addition processing of the adder, the reflection component from the direction corresponding to the reception directivity of the echo signal is emphasized.

Under the control of the processing circuitry 37, the B mode processing circuit 32 receives the echo data from the receiving circuit, performs logarithmic amplification, envelope detection processing and the like, thereby generating data (two-dimensional or three-dimensional data) whose signal intensity is represented by brightness of luminance. This data is generally called B mode data.

Under the control of the processing circuitry 37, the Doppler processing circuit 33 frequency-analyzes the phase information from the echo data from the receiving circuit, and extracts the blood flow or tissue due to the Doppler effect, thereby generating data (two-dimensional or three-dimensional data) obtained by extracting moving state information such as average speed, dispersion, power and the like for multiple points. This data is generally called Doppler data.

Under the control of the processing circuitry 37, the image generating circuit 34 generates an ultrasonic image expressed in a predetermined luminance range as image data based on the echo signal received by the ultrasonic probe 11. For example, the image generating circuit 34 generates a B mode image in which the intensity of the reflected wave is expressed in luminance from the two-dimensional B mode data generated by the B mode processing circuit 32 as the ultrasonic image. Further, the image generating circuit 34 generates, as the ultrasonic image, a color Doppler image representing moving state information from the two-dimensional Doppler data generated by the Doppler processing circuit 33 such as an average velocity image, a dispersed image, a power image, or a combined image thereof.

The image memory 35 includes memory cells in two axial directions per frame, and includes a two-dimensional memory which is a memory having the memory cells for frames. Under the control of the processing circuitry 37, the two-dimensional memory as the image memory 35 stores the ultrasonic image of one frame or the ultrasonic images frames generated by the image generating circuit 34 as two-dimensional image data.

Under the control of the processing circuitry 37, the image generating circuit 34 performs three-dimensional reconstruction on the ultrasonic image arranged in the two-dimensional memory as the image memory 35, if necessary, by interpolation processing, thereby generating an ultrasonic image as volume data in a three-dimensional memory as the image memory 35. As an interpolation processing method, a known technique is used.

The image memory 35 may include a three-dimensional memory which is a memory having memory cells in three axial directions (X-axis, Y-axis, and Z-axis direction). The three-dimensional memory as the image memory 35 stores the ultrasonic image generated by the image generating circuit 34 as volume data under the control of the processing circuitry 37.

The network interface 36 implements various information communication protocols according to the form of the network. In accordance with these various protocols, the network interface 36 connects the main body 10 with the X-ray diagnostic apparatus 50 or the like. For this connection, electrical connection or the like via an electronic network can be applied. In this embodiment, the electronic network means the whole information communication network using the telecommunication technology, and includes a local area network (LAN) of a wireless/wired hospital core and an internet network, a telephone communication network, an optical fiber communication network, a cable communication network, a satellite communication network, Wifi, Bluetooth (registered trademark), and the like.

Further, the network interface 36 may implement various protocols for non-contact wireless communication. In this case, the main body 12 can directly exchange data with the ultrasonic probe 11, for example, without going through the network.

The processing circuitry 37 means an ASIC, a programmable logic device, etc. in addition to a dedicated or general purpose central processing unit (CPU), a micro processor unit (MPU), or graphics processing unit (GPU). For the programmable logic device, for example, a simple programmable logic device (SPLD), a complex programmable logic device (CPLD), a field programmable gate array (FPGA).

Further, the processing circuitry 37 may be constituted by a single circuit or a combination of independent circuit elements. In the latter case, the internal memory 38 may be provided individually for each circuit element, or a single internal memory 38 may store programs corresponding to the functions of the circuit elements.

The internal memory 38 is constituted by a semiconductor memory element such as a random access memory (RAM), a flash memory, a hard disk, an optical disk, or the like. The internal memory 38 may be constituted by a portable medium such as a universal serial bus (USB) memory and a digital video disk (DVD). The internal memory 38 stores various processing programs (including an OS (operating system) and the like besides the application program) used in the processing circuitry 37 and data necessary for executing the programs. In addition, the OS may include a graphical user interface (GUI) which allows the operator to frequently use graphics to display information on the display 14 to the operator and can perform basic operations by the input interface 13.

The input interface 13 includes a circuit for inputting a signal from an input device operable by an ultrasonic operator D2 and an input device. The input device may be a trackball, a switch, a mouse, a keyboard, a touch pad for performing an input operation by touching an operation surface, a touch screen in which a display screen and a touch pad are integrated, a non-contact input circuit using an optical sensor, an audio input circuit, or the like. When the input device is operated by the operator, the input interface 13 generates an input signal corresponding to the operation and outputs it to the processing circuitry 37.

The display 14 is constituted by a general display output device such as a liquid crystal display or an organic light emitting diode (OLED) display. The display 14 includes a GPU (Graphics Processing Unit), a VRAM (Video RAM), and the like. Under the control of the processing circuitry 37, the display 14 displays an ultrasonic image (for example, a live image) requested for display output from the processing circuitry 37.

The position sensor 15 detects multiple positional information of the ultrasonic probe 11 in time series and outputs them to the main body 12. As the position sensor 15, there are a sensor of a type attached to the ultrasonic probe 11 and a sensor of a type provided separately from the ultrasonic probe 11. The latter sensor is an optical sensor imaging the characteristic points of the ultrasonic probe 11 to be measured from a plurality of positions, and detecting each position of the ultrasonic probe 11 on the principle of triangulation. Hereinafter, the case where the position sensor 15 is the former sensor will be described.

The position sensor 15 is attached to the ultrasonic probe 11, detects its own position data, and outputs it to the main body 12. The position data of the position sensor 15 can also be regarded as the position data of the ultrasonic probe 11. The position data of the ultrasonic probe 11 includes a position and an attitude (tilt angle) of the ultrasonic probe 11. For example, the attitude of the ultrasonic probe 11 can be detected by sequentially transmitting magnetic fields of three axes by a magnetic field transmitter (not shown) and sequentially receiving the magnetic field by the position sensor 15. Further, the position sensor 15 may be a so-called nine-axis sensor. The nine-axis sensor includes at least one of a three-axis gyro sensor for detecting the angular velocity of three axes in a three-dimensional space, a three-axis acceleration sensor for detecting accelerations of three axes in three-dimensional space, and a three-axis geomagnetic sensor for detecting three-axis geomagnetisms in three-dimensional space.

The X-ray diagnostic apparatus 50 includes a high voltage supply 51, an X-ray irradiator 52, an X-ray detector 53, an input interface 54, a display 55, a network interface 56, processing circuitry 57, an internal memory 58, a C-arm 59 (shown only in FIG. 2), and a bed 60 (shown only in FIG. 2).

The high voltage supply 51 supplies high voltage power to an X-ray tube of the X-ray irradiator 52 under the control of the processing circuitry 57.

The X-ray irradiator 52 is provided at one end of the C-arm 59. The X-ray irradiator 52 is provided with the X-ray tube (X-ray source) and a movable diaphragm device. The X-ray tube receives high-voltage power from the high-voltage supply 51 and generates X-rays according to the condition of high voltage power. Under the control of the processing circuitry 57, the movable diaphragm device movably supports diaphragm blades made of a material that shields X-rays at the X-ray irradiation port of the X-ray tube. A linear quality adjustment filter (not shown) for adjusting the quality of X-rays generated by the X-ray tube may be provided on the front face of the X-ray tube.

The X-ray detector 53 is provided at the other end of the C-arm 59 to face the X-ray irradiator 52. Under the control of the processing circuitry 57, the X-ray detector 53 can operate along the SID (Source-Image Distance) direction, that is, perform forward and backward operations. Further, under the control of the processing circuitry 57, the X-ray detector 53 can perform an operation, that is, a rotation operation, along a rotation direction around the SID direction.

The input interface 54 has a configuration equivalent to that of the input interface 13. When the input interface 54 is operated by an operator D (a doctor D1, an ultrasonic operator D2, an assistant, etc.) in the treatment room, the operation signal is sent to the processing circuitry 57.

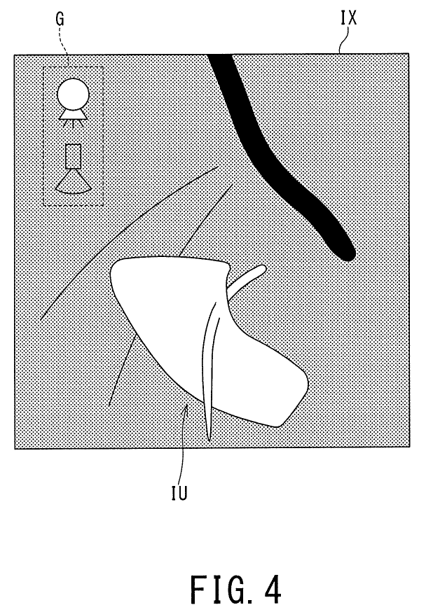

The display 55 has a configuration equivalent to that of the display 14. The display 55 displays the ultrasonic image generated according to ultrasonic imaging and the X-ray projection image generated according to X-ray imaging. For example, the display 55 displays a superimposed image (for example, shown in FIG. 4) on which an ultrasonic image is superimposed on an X-ray projection image, or displays the X-ray projection image and the ultrasonic image in parallel during the procedure.

The network interface 56 has a configuration equivalent to that of the network interface 36.

The processing circuitry 57 has a configuration equivalent to that of the processing circuitry 37.

The internal memory 58 has a configuration equivalent to that of the internal memory 38.

The C-arm 59 supports the X-ray irradiator 52 and the X-ray detector 53 to face each other. The C-arm 59 can rotate in a circular arc direction, that is, rotate in a direction of a CRA (Cranial View) and a rotation in a CAU (Caudal View) under the control of the processing circuitry 57 or in accordance with a manual operation. Further, the C-arm 59 can rotate about a fulcrum center, that is, rotate in a direction of a LAO (Left Anterior Oblique View), and a direction of a RAO (Right Anterior Oblique View) under the control of the processing circuitry 57 or in accordance with a manual operation. Conversely, the rotation of the C-arm 59 in the circular arc direction may correspond to the rotation in the direction of the LAO and the rotation in the direction of the RAO, and the rotation of the center of the fulcrum center of the C-arm 59 may correspond to the rotation in the direction of the CRA and the rotation in the direction of the CAU.

In FIG. 2, the C-arm structure included in the X-ray diagnostic apparatus 50 shows a case where the X-ray irradiator 52 is an under table positioned below the tabletop of the bed 60. However, the present invention is not limited to this case, and the X-ray irradiator 52 may be an over table located above the top plate. Further, the C-arm 59 may be replaced by an c-arm, or the Q arm may be combined.

The bed 60 includes a tabletop on which an object, for example, a patient P can be placed. Under the control of the processing circuitry 57, the tabletop can move along the X-axis direction, that is, slide in the left and right direction. Under the control of the processing circuitry 57, the tabletop can move in the Y-axis direction, that is, slide in the elevating direction. Under the control of the processing circuitry 57, the tabletop can move along the Z-axis direction, that is, slide in the cephalad direction. The tabletop can also perform a rolling operation and a tilting operation under the control of the processing circuitry 57.

Next, functions of the medical image diagnostic system 1 will be described.

The processing circuitry 37 realizes an ultrasonic imaging function U by reading out and executing a program stored in the internal memory 38 or directly incorporated in the processing circuitry 37. Hereinafter, a case where the function U functions as software will be described as an example, but the function U may be realized by a circuit such as an ASIC provided in the ultrasonic diagnostic apparatus 10.

The ultrasonic imaging function U includes a function of controlling the transmitting and receiving circuit 31, the B mode processing circuit 32, the Doppler processing circuit 33, the image generating circuit 34, and the image memory 35 to execute the ultrasonic imaging. The ultrasonic imaging function U includes a function of displaying an ultrasonic image generated according to ultrasonic imaging on the display 14 and a function of transmitting the ultrasonic image to the X-ray diagnostic apparatus 50 via the network interface 36.

The processing circuitry 57 realizes an X-ray imaging function R, a display control function Q1, a determining function Q2, and informing function Q3 by reading out and executing a program stored in the internal memory 58 or directly incorporated in the processing circuitry 57. Hereinafter, a case where the functions R and Q1 to Q3 function in software will be described as an example, but all or a part of the functions R and Q1 to Q3 may be realized by a circuit such as an ASIC provided in the X-ray diagnostic apparatus 50.

The X-ray imaging function R includes a function of controlling the high-voltage supply 51, the X-ray irradiator 52, and the X-ray detector 53 to execute an X-ray imaging. The X-ray imaging function R includes a function of displaying an X-ray image generated according to the X-ray imaging together with the ultrasonic image transmitted from the ultrasonic diagnostic apparatus 10 on the display 55. The X-ray imaging includes an X-ray imaging in a fluoroscopy mode and an X-ray imaging in a radiography mode. The radiography mode means a mode of irradiating relatively strong X-rays to obtain a clear X-ray image of contrast. The fluoroscopy mode means a mode in which relatively weak X-rays are irradiated continuously or pulsively.

The display control function Q1 acquires the ultrasonic image generated according to the ultrasonic imaging by the ultrasonic imaging function U from the ultrasonic diagnostic apparatus 10 and acquires the X-ray projection image generated according to the X-ray imaging by the X-ray imaging function R. In addition, the display control function Q1 includes a function of displaying the acquired ultrasonic image and X-ray projection image on the display 55. For example, the display control function Q1 generates a superimposed image in which the ultrasonic image is superimposed on the X-ray projection image, and displays the superimposed image on the display 55. Further, for example, the display control function Q1 displays the X-ray projection image and the ultrasonic image in parallel on the display 55. Hereinafter, it is assumed that the former, that is, the display control function Q1 generates the superimposed image.

The determining function Q2 includes a function of determining which one of the ultrasonic image and the X-ray projection image displayed on the display 55 by the display control function Q1 is live.

The informing function Q3 includes a function of informing live information indicating which one of the ultrasonic image and the X-ray projection image displayed on the display 55 is live according to the determination by the determining function Q2. For example, the informing function Q3 displays an informing image showing the live information on the display 55. In that case, the informing function Q3 displays, as the informing image, an image in which an icon corresponding to live among icons is activated on the display 55. The icons are an icon corresponding to the X-ray projection image and an icon corresponding to the ultrasonic image. Further, for example, the informing function Q3 may cause a sound indicating the live information from an operating room speaker (not shown). Alternatively, the informing function Q3 may cause a lamp (not shown) such as an LED in the operation room to light (or blink) according to the live information.

Next, the operation of the medical image diagnostic system 1 will be described. The medical image diagnostic system 1 is applied to an interventional treatment using a catheter in SHD (Structural Heart Disease). The medical image diagnostic system 1 applies not only the X-ray projection image obtained from the X-ray diagnostic apparatus 50 but also the ultrasonic image obtained from the ultrasonic diagnostic apparatus 10, and then the procedure to the patient to be described later is advanced.

For example, the medical image diagnostic system 1 is used for the catheter treatment for Mitral Regurgitation (MR) using Mitral Clip. In that case, during the procedure, an operator such as a doctor confirms the blood flow condition with the ultrasonic image of the transesophageal echocardiography (TEE) while grasping the positional relationship between a device such as a clip or the like and the heart tissue, and places that device.

### 1. First Example of Operation According to First Embodiment

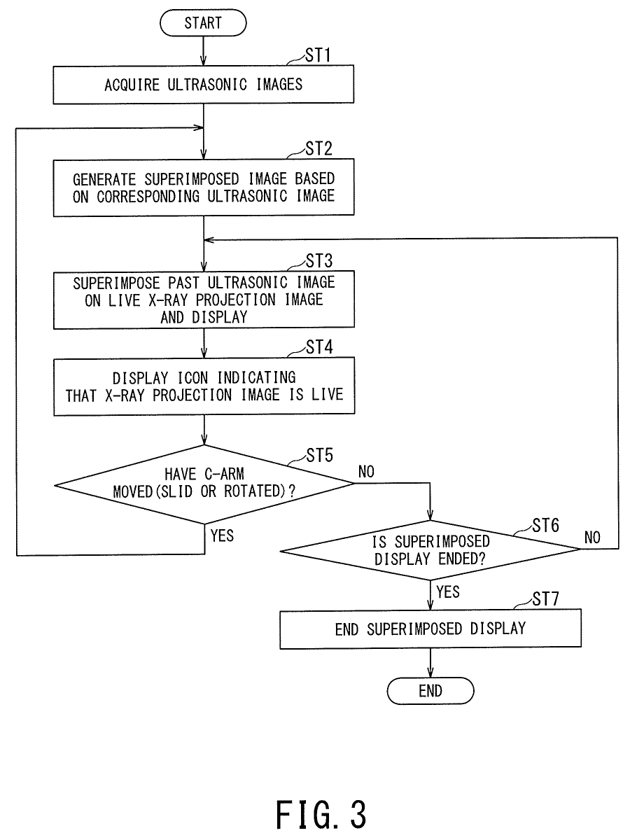

FIG. 3 is a flowchart showing a first operation example of the medical image diagnostic system 1. In FIG. 3, the reference numerals assigned “ST” with numerals indicate the respective steps of the flowchart. FIG. 4 is a diagram showing an example of a superimposed image in which live information is given as the informing image. FIGS. 3 and 4 show a case where the X-ray projection image is live.

The display control function Q1 acquires ultrasonic images relating to frames generated in the past by the ultrasonic imaging function U and stored in the image memory 35 (step ST1). The display control function Q1 generates a superimposed image in which a corresponding ultrasonic image is superimposed on the live X-ray projection image generated by the X-ray imaging function R, the corresponding ultrasonic image being out of the ultrasonic images acquired in the step ST1 (step ST2), and the superimposed image is displayed on the display 55 (step ST3).

In step ST2, the display control function Q1 specifies the position data of the ultrasonic probe 11 on the ultrasonic diagnostic apparatus 10 corresponding to the position data of the C-arm 59 etc. on the X-ray diagnostic apparatus 50. The display control function Q1 acquires an ultrasonic image corresponding to the identified position data of the ultrasonic probe 11 out of the ultrasonic images stored in the image memory 35, superimposes it on the live X-ray projection image, and displays it on the display 55.

For example, the display control function Q1 specifies a focal position of the X-ray irradiator 52 provided at one end of the C-arm 59 and an imaging direction. The imaging direction is a direction from the focus position toward the center position of the X-ray detector 53 provided at the other end of the C-arm 59. Since the position data of the ultrasonic probe 11 is associated with the ultrasonic image of each frame, the display control function Q1 acquires an ultrasonic image having position data (position and orientation). The ultrasonic image substantially coincides with the focal position on the X-ray diagnostic apparatus 50 and the imaging direction. Further, the display control function Q1 may affine transform the ultrasonic image to substantially match the focal position and the imaging direction on the X-ray diagnostic apparatus 50. The affine transformation includes translation (scaling, shearing, and rotation), linear transformation, or the like.

Although the case where the display control function Q1 acquires the ultrasonic image corresponding to the specified position data of the ultrasonic probe 11 out of the ultrasonic images stored in the image memory 35 in step ST2 will be described, but it is not limited to that case. For example, when the ultrasonic image is stored as volume data in the image memory 35, the display control function Q1 may generate the ultrasonic image corresponding to the position data of the X-ray diagnostic apparatus 50 from the volume data of a predetermined frame.

In that case, the display control function Q1 specifies the focal position of the X-ray irradiator 52 provided at one end of the C-arm 59 as a viewpoint position in the rendering (volume rendering, surface rendering, or the like) processing of the volume data of the ultrasonic image. The display control function Q1 specifies the imaging direction from the focal position toward the center position of the X-ray detector 53 provided at the other end of the C-arm 59 as a line-of-sight direction in the volume data rendering processing of the ultrasonic image. Then, the display control function Q1 performs the rendering processing on the volume data of the ultrasonic image based on the specified viewpoint position and line-of-sight direction, and superimposes it on the X-ray projection image.

The position data of the X-ray diagnostic apparatus 50 can be obtained from encoder data. The display control function Q1 obtains the encoder data from a rotary encoder attached to a roller for rotating the C-arm 59. Then, the display control function Q1 calculates position data on the C-arm 59 based on the acquired encoder data. The position data of the X-ray diagnostic apparatus 50 is not limited to the position data of the C-arm 59. For example, the position data of the X-ray diagnostic apparatus 50 may include the position data of the X-ray irradiator 52 (including the movable diaphragm device) and the X-ray detector 53. In that case, the display control function Q1 obtains the encoder data from a rotary encoder attached to a roller for operating the X-ray irradiator 52 and the X-ray detector 53 in the SID direction.

The determining function Q2 determines that the Position data among the ultrasonic image and the X-ray projection image displayed on the display 55 in step ST3 is live, then the informing function Q3 displays live information indicating that the X-ray projection image is live on the display 55 as an informing image (step ST4). FIG. 4 shows an example of the informing image to which live information G is added to the superimposed image in which a past ultrasonic image IU is superimposed on a live X-ray projection image IX. In the presentation image shown in FIG. 4, the upper icon indicating “X-ray” is set active.

When the Position data is live, it is a case where the X-ray dominates compared with the ultrasonic. When the X-ray dominates, for example, at the time of inserting the catheter from the lower limb into the coronary artery of the heart, at the time of sometimes injecting the contrast medium, at the time of checking the traveling direction of the catheter at the coronary artery bifurcation, at the time of placing a device such as Mitral Clip on the valve, or the like. Whether the X-ray is superior or not can be determined based on the operation of a foot switch for instructing X-ray irradiation.

The display control function Q1 determines whether or not the C-arm 59 on the X-ray diagnostic apparatus 50 has moved (slid or rotated) (step ST5). If it is determined as “YES” in step ST5, that is, if it is determined that the C-arm 59 has moved, the display control function Q1 generates a superimposed image in which a corresponding ultrasonic image among the ultrasonic images acquired in step ST1 is superimposed on the live X-ray projection image generated by the X-ray imaging function R (step ST2). In this way, every time the C-arm 59 moves, superimposed images based on different ultrasonic images are generated.

If it is determined as “NO” in step ST5, that is, if it is determined that the C-arm 59 has not moved, the display control function Q1 determines whether or not the superimposed display is to be ended (step ST6). If it is determined as “YES” in step ST6, that is, if it is determined to end the superimposed display, the display control function Q1 ends the superimposed display (step ST7).

If it is determined as “NO” in step ST6, that is, if it is determined not to end the superimposed display, the display control function Q1 displays a superimposed image obtained by superimposing the same ultrasonic image on the live Position data at the next timing (step ST3).

According to the first operation example of the medical image diagnostic system 1 shown in FIG. 3, the live information G indicating that the X-ray projection image among the displayed ultrasonic image and X-ray projection image is live is displayed on the display 55, so it is possible for the operator D to improve the operability during the procedure. According to the first operation example of the medical image diagnostic system 1, an appropriate ultrasonic image following the movement of the C-arm 59 is superimposed on the live Position data and displayed on the display 55, so it is possible for the operator D to improve the operability during the procedure.

Further, when the X-ray projection image is live, it is possible for the operator D to advance the catheter with the Position data in the fluoroscopic mode while referring to the blood vessel image obtained by the ultrasonic image (for example, the Doppler image), so it is possible to greatly suppress the use of the contrast medium.

### 2. Second Example of Operation According to First Embodiment

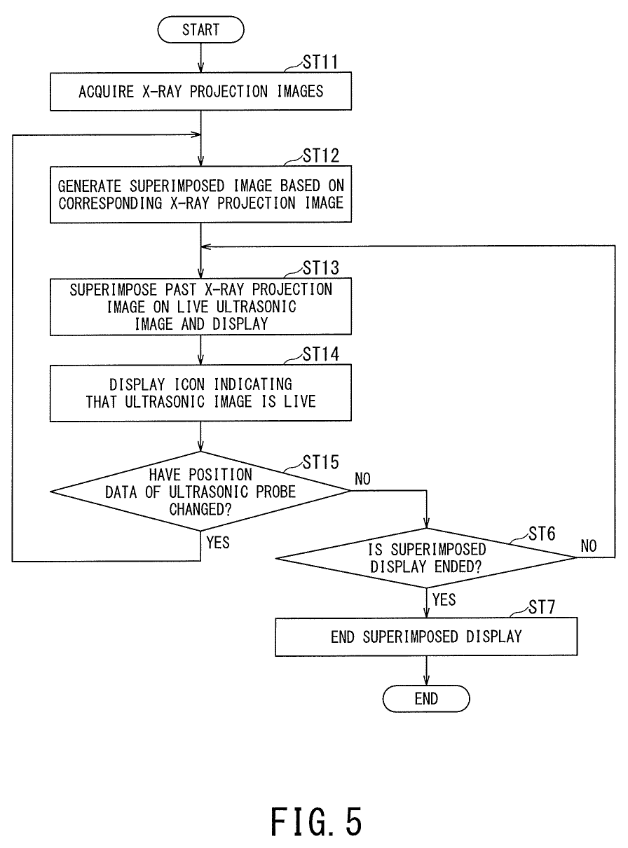

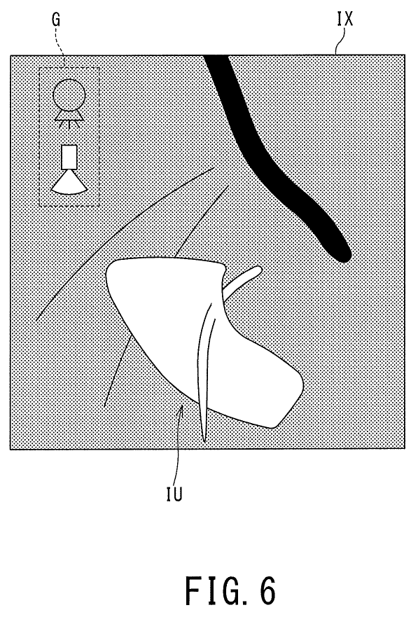

FIG. 5 is a flowchart showing a second operation example of the medical image diagnostic system 1. In FIG. 5, reference numerals assigned “ST” with numerals indicate the respective steps of the flowchart. FIG. 6 is a diagram showing an example of a superimposed image in which live information is given as the informing image. FIGS. 5 and 6 show a case where the ultrasonic image is live.

In FIG. 5, the same steps are denoted by the same reference numerals and the description thereof is omitted.

The display control function Q1 acquires X-ray projection images relating to frames generated in the past by the X-ray imaging function R and stored in the image memory (step ST11). The display control function Q1 generates a superimposed image in which a corresponding X-ray projection image among the X-ray projection images acquired in the step ST11 is superimposed on the live ultrasonic image generated by the ultrasonic imaging function U (step ST12), and the superimposed image is displayed on the display 55 (step ST13).

In step ST12, the display control function Q1 specifies the position data of the C-arm 59 etc. on the X-ray diagnostic apparatus 50. The position data corresponds to the position data of the ultrasonic probe 11 on the ultrasonic diagnostic apparatus 10. The display control function Q1 acquires an X-ray projection image corresponding to the specified position data of the C-arm 59 or the like out of the X-ray projection images stored in the internal memory 58. The display control function Q1 superimposes the live ultrasonic image on the X-ray projection image, and displays it on the display 55.

For example, the display control function Q1 specifies the position data (position and orientation) of the ultrasonic probe 11. Since the position data of the C-arm 59 etc. is associated with the X-ray projection image of each frame, the display control function Q1 acquires an X-ray projection image having the focal position and the imaging direction. The X-ray projection image substantially matches the position data on the ultrasonic diagnostic apparatus 10. Further, the display control function Q1 may affine transform the X-ray projection image to substantially match the position data on the ultrasonic diagnostic apparatus 10.

The determining function Q2 determines that the ultrasonic image among the ultrasonic image and the X-ray projection image displayed on the display 55 in step ST3 is live. The informing function Q3 display live information indicating that the ultrasonic image is live on the display 55 as the informing image (step ST14). FIG. 6 shows an example of the informing image to which live information G is added to a superimposed image in which a live ultrasonic image IU is superimposed on a past X-ray projection image IX. In the informing image shown in FIG. 6, the lower icon indicating “ultrasonic” is set active.

When the ultrasonic image is live, it is a case where the ultrasonic dominates compared with the X-ray. For example, the case where ultrasonic is dominant means a case where the catheter is inserted in the coronary artery, or a case where the clamped state of the valve after indwelling Mitral Clip is prognostically observed. Whether or not the ultrasonic is dominant may be based on a state in which an ultrasonic probe is left in the air, a state in which the ultrasonic image does not include an image of patient P that is a target, or a state in which the ultrasonic probe is in contact with a body surface. That is, it may be based on whether the ultrasonic image includes the affected part of the patient P.

The display control function Q1 determines whether or not the position data of the ultrasonic probe 11 on the ultrasonic diagnostic apparatus 10 has changed over a predetermined value (step ST15). If it is determined as “YES” in step ST15, that is, if it is determined that the position data of the ultrasonic probe 11 has changed over the predetermined value, the display control function Q1 generates a superimposed image in which a live ultrasonic image generated by the ultrasonic imaging function U is superimposed on the corresponding Position data among the X-ray projection images acquired in step ST11 (step ST12). In this way, every time the position data of the ultrasonic probe 11 changes, superimposed images based on different X-ray projection images are generated.

If it is determined as “NO” in step ST15, that is, if it is determined that the position data of the ultrasonic probe 11 has not changed, the display control function Q1 determines whether or not the superimposed display is to be ended (step ST6).

According to the second operation example of the medical image diagnostic system 1 shown in FIG. 5, the live information G indicating that the ultrasonic image among the displayed ultrasonic image and X-ray projection image is live is displayed on the display 55, so it is possible for the operator D to improve the operability during the procedure. According to the second operation example of the medical image diagnostic system 1, the live ultrasonic image is superimposed on an appropriate X-ray projection image that follows the movement of the ultrasonic probe 11 and displayed on the display 55, so it is possible for the operator D to improve the operability during the procedure.

In addition, when the ultrasonic image is live, it is possible to proceed the procedure while observing the live ultrasonic image while the operator D watches the X-ray contrast image as a reference, so it is possible for the patient P to suppress X-ray exposure.

Although an example in which non-live images are aligned and displayed on live has been described with reference to FIGS. 3 and 5, it is not limited to that case. For example, there is a case where a live image is aligned and displayed on the other live image.

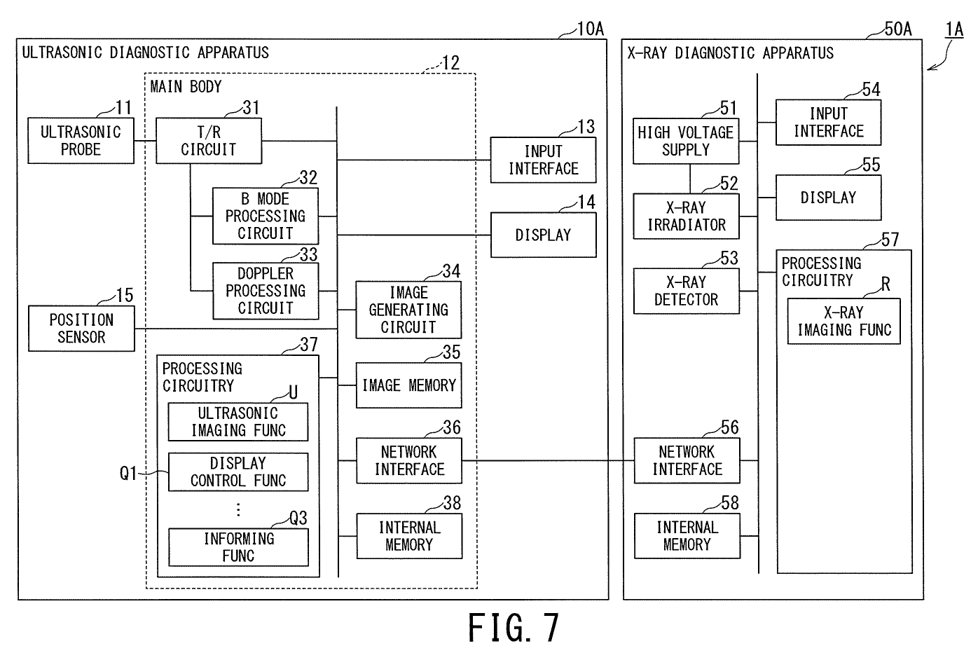

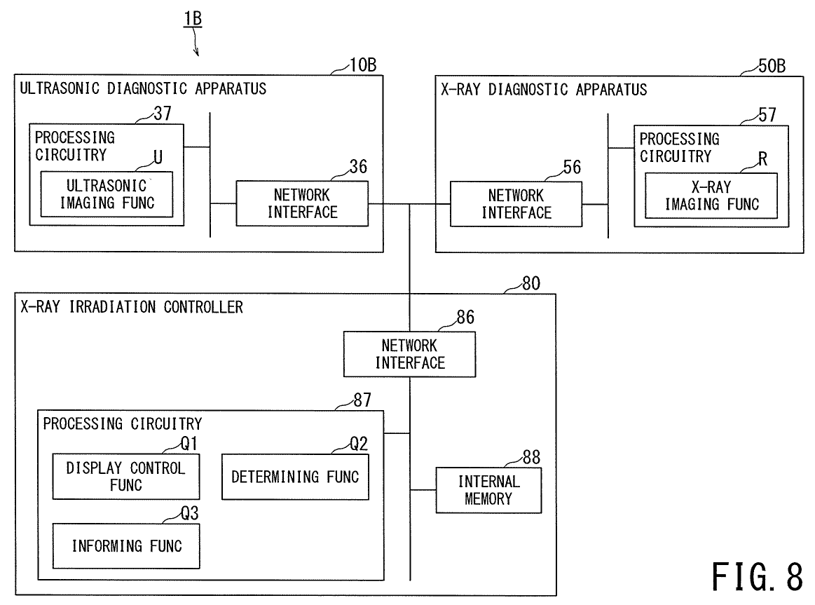

In the above description, it is described that the functions Q1 to Q3 are realized by the processing circuitry 57 of the X-ray diagnostic apparatus 50, but the present invention is not limited to this case. For example, all or part of the functions Q1 to Q3 may be realized by the processing circuitry 37 of the ultrasonic diagnostic apparatus 10, or may be realized by an apparatus other than the ultrasonic diagnostic apparatus 10 and the X-ray diagnostic apparatus 50. Hereinafter, a case where all of the functions Q1 to Q3 are realized by the processing circuitry 37 of the ultrasonic diagnostic apparatus 10 will be described with reference to FIG. 7, and a case where all of the functions Q1 to Q3 are realized by the processing circuitry of the X-ray irradiation controller other than the ultrasonic diagnostic apparatus 10 and the X-ray diagnostic apparatus 50 will be described with reference to FIG. 8.

### 3. Second Embodiment

FIG. 7 is a schematic diagram showing a configuration of a medical image diagnostic system according to a second embodiment.

FIG. 7 shows a medical image diagnostic system 1A according to a second embodiment. The medical image diagnostic system 1A includes an ultrasonic diagnostic apparatus 10A as a medical image diagnostic apparatus according to the second embodiment and an X-ray diagnostic apparatus 50A.

In FIG. 7, the same members as those in FIG. 1 are denoted by the same reference numerals, and description thereof is omitted.

The processing circuitry 37 of the ultrasonic diagnostic apparatus 10A realizes the ultrasonic imaging function U, the display control function Q1, the determining function Q2, and the informing function Q3 by executing the program. The processing circuitry 57 of the X-ray diagnostic apparatus 50A implements the X-ray imaging function R by executing a program.

The functions U, R, and Q1 to Q3 have been described in the first embodiment with reference to FIGS. 1 to 6, so that the description thereof will be omitted.

According to the medical image diagnostic system 1A shown in FIG. 7, the live information G indicating that any one of the displayed ultrasonic image and the X-ray projection image is live is displayed on the display 55, so it is possible for the operator D to improve the operability during the procedure. According to the medical image diagnostic system 1A shown in FIG. 7, the appropriate image that follows the movement of the C-arm 59 and the change in the position data of the ultrasonic probe 11 is displayed on the display 55, so it is possible for the operator D to improve the operability during the procedure.

### 4. Third Embodiment

FIG. 8 is a schematic diagram showing a configuration of a medical image diagnostic system according to a third embodiment.

FIG. 8 shows the medical image diagnostic system 1B according to the third embodiment. The medical image diagnostic system 1B includes an ultrasonic diagnostic apparatus 10B, an X-ray diagnostic apparatus 50B, and an X-ray irradiation controller 80 according to the third embodiment. The X-ray irradiation controller 80 is connected to the ultrasonic diagnostic apparatus 10B and the X-ray diagnostic apparatus 50B to be able to communicate with each other.

In FIG. 8, the same members as those in FIG. 1 are denoted by the same reference numerals, and description thereof is omitted. The ultrasonic diagnostic apparatus 10B includes an ultrasonic probe 11, a main body 12, an input interface 13, a display 14, and a position sensor 15 like the ultrasonic diagnostic apparatus 10 (shown in FIG. 1) or 10A (shown in FIG. 7), but configurations other than the network interface 36 and the processing circuit 37 are not shown. Similarly to the X-ray diagnostic apparatus 50 (shown in FIG. 1) or 50A (shown in FIG. 7), the X-ray diagnostic apparatus 50B includes a high voltage supply 51, an X-ray irradiator 52, an X-ray detector 53, an input interface 54, a display 55, a network interface 56, processing circuitry 57, and an internal memory 58, but configurations other than the network interface 56 and the processing circuitry 57 are not shown.

The X-ray irradiation controller 80 includes a network interface 86, processing circuitry 87, and an internal memory 88. The X-ray irradiation controller 80 may include an input interface having the same configuration as the input interfaces 13 or 54 (shown in FIG. 1) and a display having the same configuration as the displays 14 or 55 (shown in FIG. 1).

The processing circuitry 37 of the ultrasonic diagnostic apparatus 10B realizes the ultrasonic imaging function U by executing the program. The processing circuitry 57 of the X-ray diagnostic apparatus 50B realizes the X-ray imaging function R by executing the program.

The processing circuitry 87 of the X-ray irradiation controller 80 reads out and executes a program stored in the internal memory 88 or directly incorporated in the processing circuitry 87, thereby realizing a display control function Q1, a determining function Q2, and an informing function Q3. Hereinafter, a case where the functions Q1 to Q3 function in software will be described as an example, but one or all of the functions Q1 to Q3 may be realized by a circuit such as an ASIC provided in the X-ray irradiation controller 80.

The functions U, R, and Q1 to Q3 have been described in the first embodiment with reference to FIGS. 1 to 6, so the description thereof will be omitted.

According to the medical image diagnostic system 1B shown in FIG. 8, the live information G indicating that any one of the displayed ultrasonic image and the X-ray projection image is live is displayed on the display 55, so it is possible for the operator D to improve the operability during the procedure. According to the medical image diagnostic system 1B shown in FIG. 8, the appropriate image that follows the movement of the C-arm 59 and the change in the position data of the ultrasonic probe 11 is displayed on the display 55, so it is possible for the operator D to improve the operability during the procedure.

According to at least one of the embodiments described above, it is possible for the operator to improve the operability during the procedure.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising:
processing circuitry configured to display an ultrasonic image and a non-ultrasonic medical image to a display,
determine which of a displayed ultrasonic image and a displayed non-ultrasonic medical image is live, and
inform information indicating which any one of the displayed ultrasonic image and the displayed non-ultrasonic medical image is live in accordance with the determination.

2. The medical image diagnostic apparatus according to claim 1, wherein
the non-ultrasonic medical image is an X-ray projection image, an X-ray computed tomography (CT) image or a magnetic resonance (MR) image.

3. The medical image diagnostic apparatus according to claim 2, wherein
the non-ultrasonic medical image is the X-ray projection image.

4. The medical image diagnostic apparatus according to claim 3, wherein
the processing circuitry is configured to display an informing image indicating the information on the display.

5. The medical image diagnostic apparatus according to claim 4, wherein
the processing circuitry is configured to display, as the informing image, an image in which an icon corresponding to live among icons is activated on the display, the icons being an icon corresponding to the X-ray projection image and an icon corresponding to the ultrasonic image.

6. The medical image diagnostic apparatus according to claim 3, wherein the processing circuitry is configured to
specify, in a case where the ultrasonic image is live, position data of an arm supporting an X-ray irradiator and an X-ray detector corresponding to position data of an ultrasonic probe, and
acquire an X-ray projected image corresponding to the specified position data out of X-ray projected images stored in a memory, and
display the acquired X-ray projection image om the display.

7. The medical image diagnostic apparatus according to claim 3, wherein the processing circuitry is configured to
specify, in a case where the X-ray projection image is live, position data of an ultrasonic probe corresponding to position data of an arm supporting an X-ray irradiator and an X-ray detector, and
acquire an ultrasonic image corresponding to the specified position data out of ultrasonic images stored in a memory, and
display the acquired ultrasonic image on the display.

8. The medical image diagnostic apparatus according to claim 3, wherein the processing circuitry is configured to
generate, in a case where the X-ray projection image is live, an ultrasonic image corresponding to position data of an arm supporting an X-ray irradiator and an X-ray detector from volume data by ultrasonic imaging, and
display the ultrasonic image on the display.

9. The medical image diagnostic apparatus according to claim 3, wherein
the processing circuitry is configured to display a superimposed image obtained by superimposing the ultrasonic image on the X-ray projection image on the display.

10. The medical image diagnostic apparatus according to claim 3, further comprising:
an X-ray irradiator configured to irradiate X-rays for an X-ray imaging; and
an X-ray detector configured to detect the X-rays,
wherein
the processing circuitry is configured to control the X-ray irradiator and the X-ray detector to perform the X-ray imaging.

11. The medical image diagnostic apparatus according to claim 3, wherein
the processing circuitry is configured to further control an ultrasonic probe to control an ultrasonic imaging.

12. The medical image diagnostic apparatus according to claim 3, wherein
the processing circuitry is configured to determine that the ultrasonic image is live based on a state in which an ultrasonic probe is left in the air, a state in which the ultrasonic image does not include an image of a target, or a state in which the ultrasonic probe is in contact with a body surface.

13. The medical image diagnostic apparatus according to claim 3, wherein
the processing circuitry is configured to determine that the X-ray projection image is live based on an operation of a foot switch for instructing an X-ray irradiation.

14. An X-ray irradiation controller connected to be able to communicate mutually with an ultrasonic diagnostic apparatus and an X-ray diagnostic apparatus comprising: processing circuitry configured to
display an ultrasonic image and a non-ultrasonic medical image to a display,
determine which of a displayed ultrasonic image and a displayed non-ultrasonic medical image is live, and
inform information indicating which any one of the displayed ultrasonic image and the displayed non-ultrasonic medical image is live in accordance with the determination.
